(19) United States
(12) Patent Application Publication
(10) Pub. No.: US 2026/0114892 A1
(43) Pub. Date: Apr. 30, 2026

(54) THERMOCOUPLE FOR ULTRASONIC INSTRUMENT

(71) Applicant: Covidien LP, Mansfield, MA (US)

(72) Inventors: Richard L. CROFT, Mead, CO (US); Michael B. LYONS, Boulder, CO (US); David J. VAN TOL, Boulder, CO (US); Matthew S. COWLEY, Frederick, CO (US); Keith W. MALANG, Longmont, CO (US); Graham J. BARRETT, Denver, CO (US)

(21) Appl. No.: 19/123,665

(22) PCT Filed: Oct. 20, 2023
(86) PCT No.: PCT/IB2023/060620

§ 371 Date: Apr. 23, 2025

Related U.S. Application Data
(60) Provisional application No. 63/419,469, filed on Oct. 26, 2022.

(51) Int. Cl. A61B 17/32; A61B 17/00

(52) U.S. Cl. CPC A61B 17/320092; A61B 2017/00092; A61B 2017/320094

(57) ABSTRACT

An ultrasonic surgical instrument (10) includes a housing (112) having an elongated shaft (150) extending therefrom with an ultrasonic transducer (140) disposed therein. The ultrasonic transducer (140) is operably coupled to a waveguide (154). An ultrasonic blade (162) made from a first metal is operably coupled to the waveguide (154) and extends therefrom. The ultrasonic blade (162) is configured to vibrate upon activation of the ultrasonic transducer (140) to treat tissue. A second, dissimilar metal (500) is disposed on the ultrasonic blade. A thermocouple (600) is formed by a first electrical lead (520) extending from a reference junction (153) and a second electrical lead (510) coupled to the second, dissimilar metal (500). The thermocouple (600) is configured to detect a temperature difference between reference junction (153) and the portion of the blade (162) supporting the second, dissimilar metal (500). The thermocouple (600) derives a temperature at the second, dissimilar metal (500) from a voltage generated at a junction between the first metal and the second, dissimilar metal and a temperature at the reference junction (153).

### FIELD

The present disclosure relates to surgical instruments and, more particularly, to ultrasonic surgical instruments for performing multiple surgical tasks.

### BACKGROUND

Ultrasonic surgical instruments and systems utilize ultrasonic energy, i.e., ultrasonic vibrations, to treat tissue. More specifically, ultrasonic surgical instruments and systems utilize mechanical vibration energy transmitted at ultrasonic frequencies to treat tissue. An ultrasonic surgical device may include, for example, an ultrasonic blade and a clamp mechanism to enable clamping of tissue against the blade. Ultrasonic energy transmitted to the blade causes the blade to vibrate at very high frequencies, which allows for heating tissue to treat tissue clamped against or otherwise in contact with the blade. Ultrasonic blades may also be utilized for performing other surgical tasks such as, for example, dissection, scoring, otomies, etc.

As mentioned above, during use, the vibration of the blade at the ultrasonic frequencies causes the blade and the various other elements in close proximity thereof, e.g., the waveguide, to heat to very high temperatures requiring careful observation and placement of the instrument until components have had an opportunity to cool. As a result, the temperature of the blade and the components proximal thereto play an important role with instrument handling during activation and post activation. Moreover, and due to the particular nature of ultrasonic instruments, conventional temperature measurements of the blade and the components near the blade, e.g., waveguide, are often difficult or unreliable due to the high frequency vibrations.

### SUMMARY

As used herein, the term “distal” refers to the portion that is described which is further from an operator (whether a human surgeon or a surgical robot), while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, and/or other variations, up to and including plus or minus 10 percent. Further, any or all of the aspects described herein, to the extent consistent, may be used in conjunction with any or all of the other aspects described herein.

Provided in accordance with aspects of the present disclosure is an ultrasonic surgical instrument which includes a housing having an elongated shaft extending therefrom. An ultrasonic transducer having a waveguide operably coupled thereto, the waveguide is configured to extend through the elongated shaft, the ultrasonic transducer adapted to couple to an energy source to allow selective activation thereof. An ultrasonic blade made from a first metal is operably coupled to the waveguide and is configured to extend therefrom, the ultrasonic blade configured to vibrate upon activation of the ultrasonic transducer to treat tissue. A second, dissimilar metal is disposed on a portion of the ultrasonic blade. A thermocouple is formed by a first electrical lead extending from a reference junction at the proximal end portion of the waveguide and a second electrical lead coupled to the second, dissimilar metal, the thermocouple is configured to detect a temperature difference between the proximal end portion of the waveguide and the portion of the blade supporting the second, dissimilar metal, the thermocouple deriving a temperature at the second, dissimilar metal from a voltage generated at a junction between the first metal and the second, dissimilar metal and a temperature at the reference junction.

In aspects according to the present disclosure, the second, dissimilar metal is deposited onto the blade via metal or plasma deposition, chemical vapor deposition, printing, spraying, sintering, curing, thermal spray (HVOF), 3D printing, silk screen, or applying metal inks or paints.

In aspects according to the present disclosure, the ultrasonic instrument includes a handle operably coupled to the housing and configured to selectively move relative thereto to pivot a jaw member relative to the ultrasonic blade to clamp tissue therebetween.

In aspects according to the present disclosure, the housing includes circuitry that converts the voltage obtained from the temperature difference to an actual temperature on the blade at the junction with the second, dissimilar metal, the circuitry communicating with a display panel disposed on the housing for displaying the temperature. In other aspects according to the present disclosure, the circuitry converts the voltage and communicates to the display panel in real time.

Provided in accordance with other aspects of the present disclosure is an ultrasonic surgical instrument which includes a housing having an elongated shaft extending therefrom. An ultrasonic transducer including a waveguide made from a first metal is operably coupled thereto and configured to extend through the elongated shaft, the ultrasonic transducer adapted to couple to an energy source to allow selective activation thereof. An ultrasonic blade is operably coupled to the waveguide and is configured to extend therefrom, the ultrasonic blade configured to vibrate upon activation of the ultrasonic transducer to treat tissue. A plurality of second, dissimilar metals is disposed along the waveguide and extends proximally from the ultrasonic blade. A thermocouple is formed by a first electrical lead from a reference junction operably associated with the housing and a second electrical lead coupled to each second, dissimilar metal, the thermocouple is configured to detect a temperature difference between the reference junction and the portion of the blade supporting each second, dissimilar metal. The thermocouple derives a temperature at each of the plurality of second, dissimilar metals from a voltage generated at a junction between the first metal and each of the plurality of second, dissimilar metals and a temperature at the reference junction.

In aspects according to the present disclosure, the plurality of second, dissimilar metals is deposited onto the waveguide via metal or plasma deposition, chemical vapor deposition, printing, spraying, sintering, curing, thermal spray (HVOF), 3D printing, silk screen, or applying metal inks or paints.

In aspects according to the present disclosure, the ultrasonic instrument includes a handle operably coupled to the housing and configured to selectively move relative thereto to pivot a jaw member relative to the ultrasonic blade to clamp tissue therebetween.

In aspects according to the present disclosure, the housing includes circuitry that converts the voltage obtained from the temperature difference to an actual temperature on the waveguide at each junction on the waveguide supporting the plurality of second, dissimilar metals, the circuitry communicating with a display panel disposed on the housing for displaying the temperature. In other aspects according to the present disclosure, the circuitry converts the voltage and communicates to the display panel in real time.

In aspects according to the present disclosure, one or more of the plurality of second, dissimilar metals is disposed on a node of the waveguide.

In aspects according to the present disclosure, each of the plurality of second, dissimilar metals is disposed on a node of the waveguide.

Provided in accordance with other aspects of the present disclosure is a method of determining a temperature of a blade of an ultrasonic surgical instrument that includes: electrically coupling a thermocouple across a first electrical lead extending from a proximal end portion of a waveguide integrally associated with an ultrasonic blade and a second electrical lead coupled to a second, dissimilar metal disposed atop the ultrasonic blade; obtaining a reference temperature at a reference junction; activating a transducer to energize the waveguide to vibrate the ultrasonic blade made from a first metal to treat tissue proximate the ultrasonic blade; utilizing the thermocouple to detect a temperature difference between the proximal end portion of the waveguide and the portion of the blade supporting the second, dissimilar metal on the blade, the thermocouple deriving a temperature at the second, dissimilar metal from a voltage generated at a junction between the first metal and the of second, dissimilar metal and a temperature at the reference junction; and utilizing the temperature of the portion of the blade supporting the second, dissimilar metal for safety, storage, and/or handling.

In aspects according to the present disclosure, the method further includes depositing the second, dissimilar metal atop the blade via metal or plasma deposition, chemical vapor deposition, printing, spraying, sintering, curing, thermal spray (HVOF), 3D printing, silk screen, or applying metal inks or paints.

In aspects according to the present disclosure, a housing includes circuitry that includes the thermocouple, the circuitry configured to convert the voltage obtained from the temperature difference to an actual temperature on the blade at the portion of the blade supporting the second, dissimilar metal and wherein the method further includes: communicating with a display panel disposed on the housing for displaying the temperature. In other aspects according to the present disclosure, wherein the circuitry converts the voltage and communicates to the display panel in real time.

### BRIEF DESCRIPTION OF THE DRAWINGS

The above and other aspects and features of the present disclosure will become more apparent in light of the following detailed description when taken in conjunction with the accompanying drawings wherein like reference numerals identify similar or identical elements.

FIG. 1 is a side view of a surgical system provided in accordance with the present disclosure including a surgical instrument, a surgical generator, and, in aspects, a return electrode device;

FIG. 2 is perspective view of another surgical system provided in accordance with the present disclosure including a surgical instrument incorporating an ultrasonic generator, a power source, and, in aspects, an electrosurgical generator therein;

FIG. 3 is a schematic illustration of a robotic surgical system provided in accordance with the present disclosure;

FIG. 4 is a longitudinal, cross-sectional view of a distal end portion of either of the surgical instruments of FIG. 1 or 2 including a waveguide configured to support an ultrasonic blade at a distal end thereof;

FIG. 5A is schematic illustration of a waveguide for use with either of the instruments of FIGS. 1 and 2 having a blade extending therefrom made from a first metal having a second, dissimilar metal deposited thereon, the second, dissimilar metal electrically forming a thermocouple with a proximal end of the waveguide to recognize a voltage upon a temperature difference therebetween;

FIG. 5B is a schematic illustration of a waveguide for use with either of the instruments of FIGS. 1 and 2 made from a first metal having a second, dissimilar metal deposited near a distal end thereof, the second, dissimilar metal electrically forming a thermocouple with a proximal end of the waveguide to recognize a voltage upon a temperature difference therebetween; and

FIG. 6 is schematic illustration of a waveguide for use with either of the instruments of FIGS. 1 and 2 made from a first metal having a plurality of second, dissimilar metals deposited near a distal end and extending proximally relative thereto, each of the second, plurality of dissimilar metals electrically forming a thermocouple with a proximal end of the waveguide to recognize a voltage upon a temperature difference therebetween.

### DETAILED DESCRIPTION

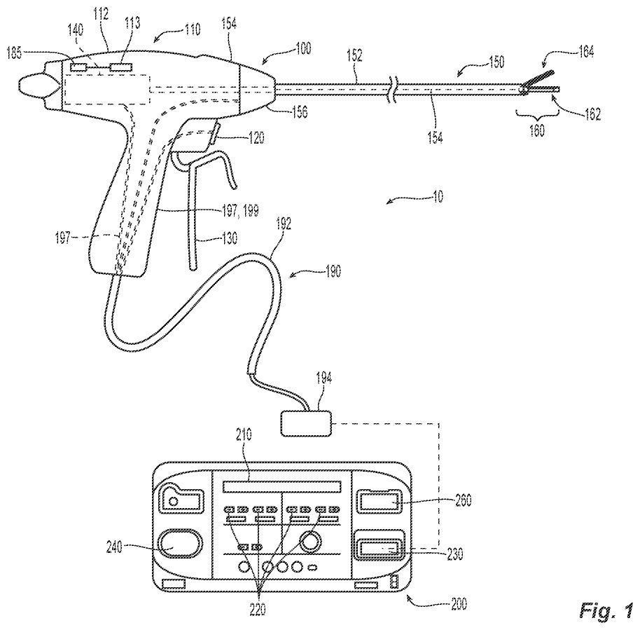

Referring to FIG. 1, a surgical system provided in accordance with aspects of the present disclosure is shown generally identified by reference numeral 10 including a surgical instrument 100, a surgical generator 200. Surgical instrument 100 includes a handle assembly 110, an elongated assembly 150 extending distally from handle assembly 110, an end effector assembly 160 disposed at a distal end of elongated assembly 150, and a cable assembly 190 operably coupled with handle assembly 110 and extending therefrom for connection to surgical generator 200.

Surgical generator 200 includes a display 210, a plurality of user interface features 220, e.g., buttons, touch screens, switches, etc., an ultrasonic plug port 230. As an alternative to plural dedicated ports 230-260, one or more common ports (not shown) may be configured to act as any two or more of ports 230-260. Surgical generator 200 is configured to produce ultrasonic drive signals for output through ultrasonic plug port 230 to surgical instrument 100 to activate surgical instrument 100 in the ultrasonic mode.

Continuing with reference to FIG. 1, handle assembly 110 includes a housing 112, an activation button 120, and a clamp trigger 130. Housing 112 is configured to support an ultrasonic transducer 140. Ultrasonic transducer 140 may be permanently engaged within housing 112 or removable therefrom. Ultrasonic transducer 140 includes a piezoelectric stack other suitable ultrasonic transducer components electrically coupled to surgical generator 200, e.g., via one or more of first electrical lead wires 197, to enable communication of ultrasonic drive signals to ultrasonic transducer 140 to drive ultrasonic transducer 140 to produce ultrasonic vibration energy that is transmitted along a waveguide 154 of elongated assembly 150 to blade 162 of end effector assembly 160 of elongated assembly 150, as detailed below. Feedback and/or control signals may likewise be communicated between ultrasonic transducer 140 and surgical generator 200. Ultrasonic transducer 140, more specifically, may include a stack of piezoelectric elements secured, under pre-compression between proximal and distal end masses or a proximal end mass and an ultrasonic horn with first and second electrodes electrically coupled between piezoelectric elements of the stack of piezoelectric elements to enable energization thereof to produce ultrasonic energy. However, other suitable ultrasonic transducer configurations, including plural transducers and/or non-longitudinal, e.g., torsional, transducers are also contemplated.

Activation button 120 is disposed on housing 112 and coupled to or between ultrasonic transducer 140 and/or surgical generator 200, e.g., via one or more of electrical lead wires 197, to enable activation of ultrasonic transducer 140 in response to depression of activation button 120. In some configurations, activation button 120 may include an ON/OFF switch. In other configurations, activation button 120 may include multiple actuation switches to enable activation from an OFF position to different actuated positions corresponding to different activation settings, e.g., a first actuated position corresponding to a first activation setting (such as a LOW power or tissue sealing setting) and a second actuated position corresponding to a second activation setting (such as a HIGH power or tissue transection setting). In still other configurations, separate activation buttons may be provided, e.g., a first actuation button for activating a first activation setting and a second activation button for activating a second activation setting. Additional activation buttons, sliders, wheels, etc. are also contemplated to enable control of various different activation settings from housing 112.

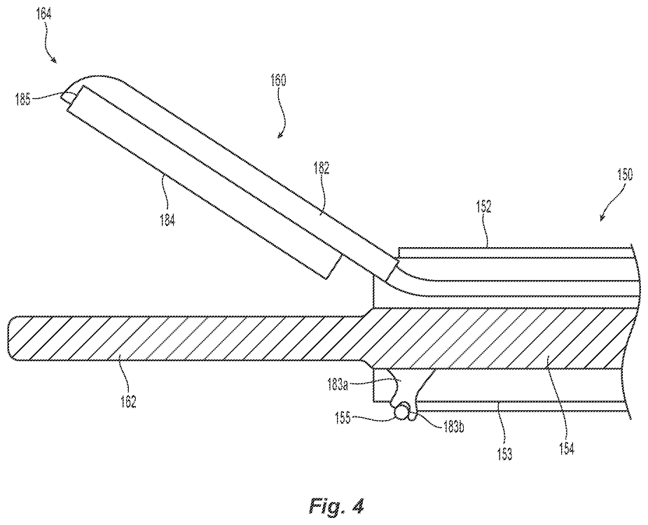

Elongated assembly 150 of surgical instrument 100 includes an outer drive sleeve 152, an inner support sleeve 153 (FIG. 4) disposed within outer drive sleeve 152, a waveguide 154 extending through inner support sleeve 153 (FIG. 4), a drive assembly (not shown), a rotation knob 156, and an end effector assembly 160 including a blade 162 and a jaw member 164. Rotation knob 156 is rotatable in either direction to rotate elongated assembly 150 in either direction relative to handle assembly 110. The drive assembly operably couples a proximal portion of outer drive sleeve 152 to clamp trigger 130 of handle assembly 110. A distal portion of outer drive sleeve 152 is operably coupled to jaw member 164 and a distal end of inner support sleeve 153 (FIG. 4) pivotably supports jaw member 164. As such, clamp trigger 130 is selectively actuatable to thereby move outer drive sleeve 152 about inner support sleeve 153 (FIG. 4) to pivot jaw member 164 relative to blade 162 of end effector assembly 160 from a spaced apart position to an approximated position for clamping tissue between jaw member 164 and blade 162. The configuration of outer and inner sleeves 152, 153 (FIG. 4) may be reversed, e.g., wherein outer sleeve 152 is the support sleeve and inner sleeve 153 (FIG. 4) is the drive sleeve. Other suitable drive structures as opposed to a sleeve are also contemplated such as, for example, drive rods, drive cables, drive screws, etc.

Referring still to FIG. 1, the drive assembly may be tuned to provide a jaw clamping force, or jaw clamping force within a jaw clamping force range, to tissue clamped between jaw member 164 and blade 162 or may include a force limiting feature whereby the clamping force applied to tissue clamped between jaw member 164 and blade 162 is limited to a particular jaw clamping force or a jaw clamping force within a jaw clamping force range.

Waveguide 154, as noted above, extends from handle assembly 110 through inner sleeve 153 (FIG. 4). Waveguide 154 includes blade 162 disposed at a distal end thereof. Blade 162 may be integrally formed with waveguide 154, separately formed and subsequently attached (permanently or removably) to waveguide 154, or otherwise operably coupled with waveguide 154. Waveguide 154 and/or blade 162 may be formed from titanium, a titanium alloy, or other suitable electrically conductive material(s), although non-conductive materials are also contemplated. Waveguide 154 includes a proximal connector (not shown), e.g., a threaded male connector, configured for engagement, e.g., threaded engagement within a threaded female receiver, of ultrasonic transducer 140 such that ultrasonic motion produced by ultrasonic transducer 140 is transmitted along waveguide 154 to blade 162 for treating tissue clamped between blade 162 and jaw member 164 or positioned adjacent to blade 162.

Cable assembly 190 of surgical instrument 100 includes a cable 192 and an ultrasonic plug 194. Ultrasonic plug 194 is configured for connection with ultrasonic plug port 230 of surgical generator 200.

Electrical lead wires 197 electrically coupled to ultrasonic plug 194 extend through cable 192 and into handle assembly 110 for electrical connection to ultrasonic transducer 140 and/or activation button 120 to enable the selective supply of ultrasonic drive signals from surgical generator 200 to ultrasonic transducer 140 upon activation of activation button 120 in an ultrasonic mode.

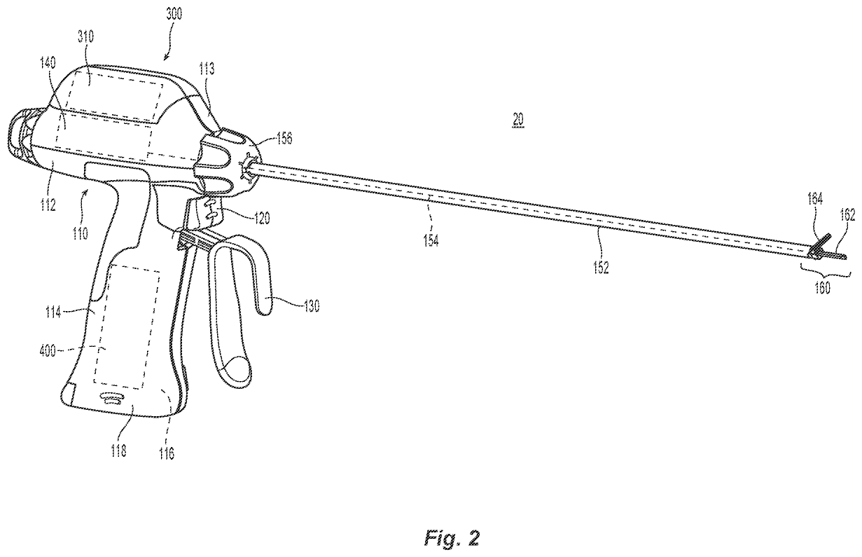

As an alternative to a remote generator 200, surgical system 10 may be at least partially cordless in that it incorporates an ultrasonic generator and/or a power source, e.g., a battery, thereon or therein. In this manner, the connections from surgical instrument 100 to external devices, e.g., generator(s) and/or power source(s), are reduced or eliminated. More specifically, with reference to FIG. 2, another surgical system in accordance with the present disclosure is shown illustrated as a surgical instrument 20 supporting an ultrasonic generator 310, a power source (e.g., battery assembly 400), and, in aspects thereon or therein. Surgical instrument 20 is similar to surgical instrument 100 (FIG. 1) and may include any of the features thereof except as explicitly contradicted below. Accordingly, only differences between surgical instrument 20 and surgical instrument 100 (FIG. 1) are described in detail below while similarities are omitted or summarily described.

Housing 112 of surgical instrument 20 includes a body portion 113 and a fixed handle portion 114 depending from body portion 113. Body portion 113 of housing 112 is configured to support an ultrasonic transducer and generator assembly (“TAG”) 300 including ultrasonic generator 310 and ultrasonic transducer 140. TAG 300 may be permanently engaged with body portion 113 of housing 112 or removable therefrom.

Fixed handle portion 114 of housing 112 defines a compartment 116 configured to receive battery assembly 400 and a door 118 configured to enclose compartment 116. An electrical connection assembly (not shown) is disposed within housing 112 and serves to electrically couple activation button 120, ultrasonic generator 310 of TAG 300, and battery assembly 400 with one another when TAG 300 is supported on or in body portion 113 of housing 112 and battery assembly 400 is disposed within compartment 116 of fixed handle portion 114 of housing 112, thus enabling activation of surgical instrument 20 in an ultrasonic mode in response to appropriate actuation of activation button 120.

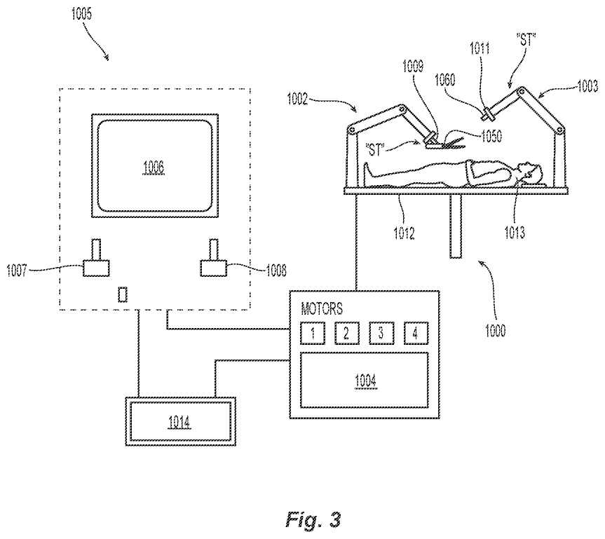

Turning to FIG. 3, a robotic surgical system in accordance with the aspects and features of the present disclosure is shown generally identified by reference numeral 1000. For the purposes herein, robotic surgical system 1000 is generally described. Aspects and features of robotic surgical system 1000 not germane to the understanding of the present disclosure are omitted to avoid obscuring the aspects and features of the present disclosure in unnecessary detail.

Robotic surgical system 1000 generally includes a plurality of robot arms 1002, 1003; a control device 1004; and an operating console 1005 coupled with control device 1004. Operating console 1005 may include a display device 1006, which may be set up in particular to display three dimensional images; and manual input devices 1007, 1008, by means of which a person (not shown), for example a surgeon, may be able to telemanipulate robot arms 1002, 1003 in a first operating mode. Robotic surgical system 1000 may be configured for use on a patient 1013 lying on a patient table 1012 to be treated in a minimally invasive manner. Robotic surgical system 1000 may further include a database 1014, in particular coupled to control device 1004, in which are stored, for example, pre-operative data from patient 1013 and/or anatomical atlases.

Each of the robot arms 1002, 1003 may include a plurality of members, which are connected through joints, and an attaching device 1009, 1011, to which may be attached, for example, a surgical tool “ST” supporting an end effector 1050, 1060. One of the surgical tools “ST” may be surgical instrument 100 (FIG. 1), surgical instrument 20 (FIG. 2), or any other suitable surgical instrument 20 configured for use in an ultrasonic mode wherein manual actuation features, e.g., actuation button 120 (FIG. 1), clamp lever 130 (FIG. 1), etc., are replaced with robotic inputs. In such configurations, robotic surgical system 1000 may include or be configured to connect to an ultrasonic generator and/or a power source. The other surgical tool “ST” may include any other suitable surgical instrument, e.g., an endoscopic camera, other surgical tool, etc. Robot arms 1002, 1003 may be driven by electric drives, e.g., motors, that are connected to control device 1004. Control device 1004 (e.g., a computer) may be configured to activate the motors, in particular by means of a computer program, in such a way that robot arms 1002, 1003, their attaching devices 1009, 1011, and, thus, the surgical tools “ST” execute a desired movement and/or function according to a corresponding input from manual input devices 1007, 1008, respectively. Control device 1004 may also be configured in such a way that it regulates the movement of robot arms 1002, 1003 and/or of the motors.

Referring back to FIG. 4, end effector assembly 160 as noted above, includes blade 162 and jaw member 164. Blade 162 typically defines a linear configuration, but in some instances may define a curved configuration, or may define any other suitable configuration, e.g., straight and/or curved surfaces, portions, and/or sections; one or more convex and/or concave surfaces, portions, and/or sections; etc. With respect to curved configurations, blade 162 may be curved in any direction relative to jaw member 164 such that the distal tip of blade 162 is curved towards jaw member 164, away from jaw member 164, or laterally (in either direction) relative to jaw member 164. Further, blade 162 may be formed to include multiple curves in similar directions, multiple curves in different directions within a single plane, and/or multiple curves in different directions in different planes. In addition, blade 162 may additionally or alternatively be formed to include any suitable features, e.g., a tapered configuration, various different cross-sectional configurations along its length, cut outs, indents, edges, protrusions, straight surfaces, curved surfaces, angled surfaces, wide edges, narrow edges, and/or other features.

Blade 162 may define a polygonal, rounded polygonal, or any other suitable cross-sectional configuration(s). Waveguide 154 or at least the portion of waveguide 154 proximally adjacent blade 162, may define a cylindrical shaped configuration. Plural tapered surfaces (not shown) may interconnect the cylindrically shaped waveguide 154 with the polygonal (rounded edge polygonal, or other suitable shape) configuration of blade 162 to define smooth transitions between the body of waveguide 154 and blade 162.

Blade 162 may be wholly or selectively coated with a suitable material, e.g., a non-stick material, an electrically insulative material, an electrically conductive material, combinations thereof, etc. Suitable coatings and/or methods of applying coatings include but are not limited to Teflon®, polyphenylene oxide (PPO), deposited liquid ceramic insulative coatings; thermally sprayed coatings, e.g., thermally sprayed ceramic; Plasma Electrolytic Oxidation (PEO) coatings; anodization coatings; sputtered coatings, e.g., silica; ElectroBond® coating available from Surface Solutions Group of Chicago, IL, USA; or other suitable coatings and/or methods of applying coatings.

Jaw member 164 of end effector assembly 160 includes more rigid structural body 182 and may include a more compliant jaw liner 184. Structural body 182 may be formed from various materials depending upon its desired purpose, electrically conductive materials, thermally conductive materials, electrically insulative materials or combinations of the same.

Structural body 182 includes a pair of proximal flanges 183a that are pivotably coupled to the inner support sleeve 153 via receipt of pivot bosses (not shown) of proximal flanges 183a within corresponding openings (not shown) defined within the inner support sleeve 153 and operably coupled with outer drive sleeve 152 via a drive pin 155 secured relative to outer drive sleeve 152 and pivotably received within apertures 183b defined within proximal flanges 183a. As such, sliding of outer drive sleeve 152 about inner support sleeve 153 pivots jaw member 164 relative to blade 162 from a spaced apart position to an approximated position to clamp tissue between jaw liner 184 of jaw member 164 and blade 162.

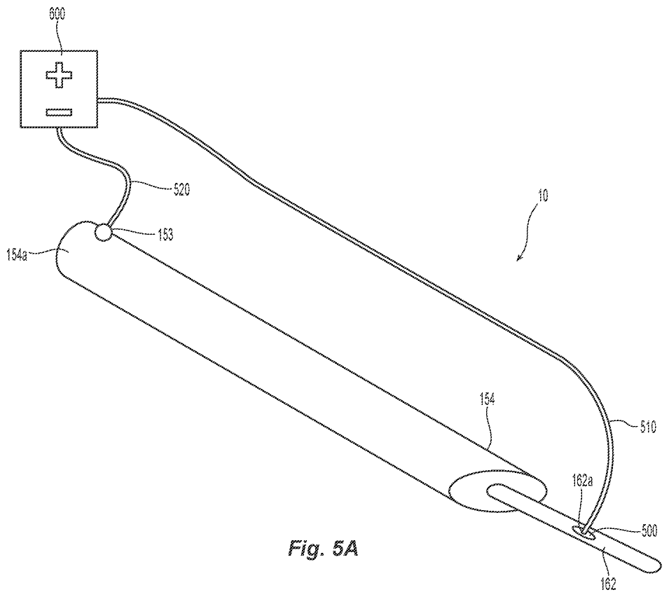

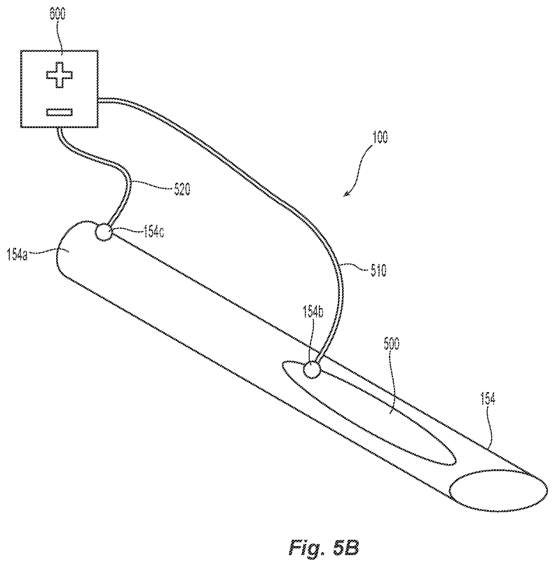

Referring to FIG. 5A, during use of an ultrasonic instrument 100 for dissection (or other tissue treatment such as coagulation or tissue sealing), the blade 162 vibrates with enough energy that it can reach temperatures in excess of 300° C. when that energy is delivered to tissue pressed against the blade 162. As can be appreciated, measuring the temperature of the blade 162 prior to, during and after activation provides important feedback to the surgeon when using and handling the ultrasonic instrument 10. Adding a traditional temperature probe on the blade 162 (to monitor temperature) in this instance would be unreliable due to the vibrational characteristics of the blade 162.

FIG. 5A shows one example of a blade 162 according to the present disclosure which includes a layer of metal 500 which is dissimilar to that of the blade 162 disposed at a point 162a along the length thereof. A first lead 510 is electrically attached to the metal 500 at one end thereof and extends at an opposite end thereof to a first potential of a thermocouple junction 600. A second lead 520 is electrically attached to a reference junction 153 and extends to a second potential of the thermocouple junction 600. Typically, the reference junction 153 (or another point within the housing 12) is held at a constant or reliable temperature to yield a constant voltage which is later used to derive the temperature at the metal 500 at the blade 162 after activation or during cool down (in real time) as explained below.

The metal 500 may be disposed on the blade 162 in any fashion known in the art. In embodiments, the metal 500 is deposited via metal or plasma deposition, chemical vapor deposition, printing, spraying, sintering, curing, thermal spray (HVOF), 3D printing, silk screen, applying metal inks or paints or similar such processes to form a thin layer of metal 500 atop the blade 162 that is electrically coupled to the first lead 510. By coupling a thermocouple 600 between two dissimilar metals, e.g., the type of metal 500 deposited atop the blade 162, the temperature difference may be measured between the two metals via the Seebeck effect.

The Seebeck effect is a phenomenon in which a temperature difference between two dissimilar electrical conductors, i.e., metals, produces a voltage difference between the two substances. Thus, the temperature difference at any point 162a along the blade 162 may be measured and compared to the known of constant temperature of reference junction 153. Other known or constant temperature junctions on the instrument 10 may be coupled to the thermocouple 600 and may be utilized for this purpose. Even if the temperature differences are small, e.g., fractions of a degree, the small voltage differences are picked up at the thermocouple 600 and easily observed. Monitoring the temperature of the portion of the blade 162 at metal 500 allows the surgeon to more safely handle the instrument 10 around delicate tissues, rest the instrument 10 between activations and/or store the instrument 10 while utilizing other instruments. In addition, ultrasonic vibrations will not affect the ability of the thermocouple 600 to measure the voltage difference across the two dissimilar metals (blade 162 and metal 500).

The metal 500 is chosen to have a Seebeck coefficient dissimilar to that of the ultrasonic blade 162. By joining these dissimilar metals, a thermocouple is formed at that point 162a. That thermocouple generates a voltage related to the temperature difference between a) the junction between the two metals and b) the connection of these metals to the sensing circuit or thermocouple junction 600. A measurement of the temperature of the blade 162 is calculated or otherwise derived from the generated voltage and a known temperature at the reference junction.

Using the Seebeck effect, the surgeon can determine the temperature of the blade 162 prior to, during and after activation and as the blade 162 cools. Moreover, applying the metal 500 onto the blade and simply measuring the voltage will remain reliable during activation and ultrasonic vibration of the blade 162.

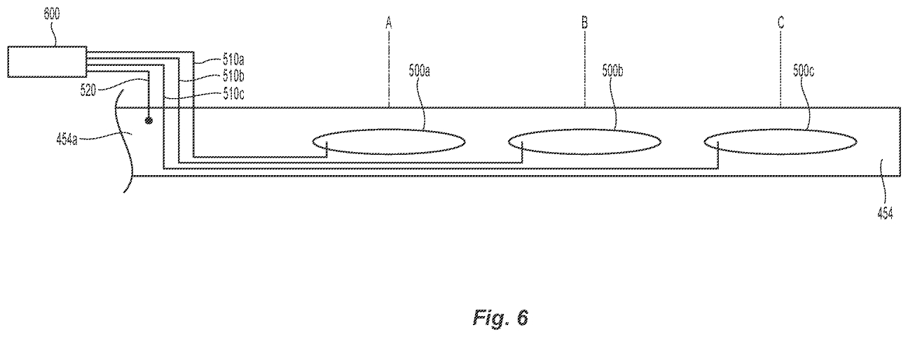

Referring now to FIGS. 5B and 6, as mentioned above during use of an ultrasonic instrument 100 for dissection (or other tissue treatment such as coagulation or tissue sealing), the blade 162 vibrates with enough energy that it can reach temperatures in excess of 300° C. when that energy is delivered to tissue pressed against the blade 162. As a result, other components integrally associated with the blade 162, e.g., the waveguide 154, or proximal to the blade 162, can also reach significantly high temperatures during tissue treatment, especially over prolonged surgical conditions. As various factors may contribute to residual heat conduction to certain components under different conditions, a surgeon may not be aware of a given temperature of a particular component in certain instances.

For example, ultrasonic surgical instruments typically transmit residual heat along the waveguide 154 which, in some instances, can come into contact with tissue and organs when the surgeon is manipulating the instrument 100 for dissection around a surgical cavity between uses. Although, a blade 162 may cool down rapidly, this does not necessarily apply to the waveguide 154. Adding a traditional temperature probe on the waveguide 154 (to monitor temperature) in this instance would be unreliable due to the vibrational characteristics of the waveguide 154. As a result, the temperature of the waveguide 154 during and after tissue treatment can become a concern when manipulating the instrument 100.

FIG. 5B shows an embodiment of a waveguide 154 according to the present disclosure which includes a layer of metal 500 which is dissimilar to that of the waveguide 154 disposed at a point 154b along the length thereof. A first lead 510 is electrically attached to the metal 500 at one end thereof and extends at an opposite end thereof to a first potential of a thermocouple junction 600. A second lead 520 is electrically attached a reference point 154c at the proximal end 154a of the waveguide 154 and extends to a second potential of the thermocouple junction 600. Typically, the reference point 154c (or another point within the housing 12 or anywhere along the waveguide 154) is held at a constant or reliable temperature to yield a constant voltage which is later used to derive the temperature at the metal 500 after activation or during cool down (in real time) as explained below.

The metal 500 may be disposed on the waveguide 154 in any fashion known in the art as described above. By coupling a thermocouple 600 between two dissimilar metals, e.g., the type of metal 500 deposited atop the metal waveguide 154, the temperature difference may be measured between the two metals via the Seebeck effect.

Monitoring the temperature of the portion of the waveguide 154 at metal 500 allows the surgeon to more safely handle the instrument 100 around delicate tissues, rest the instrument 100 between activations and/or store the instrument 100 while utilizing other instruments. In addition, ultrasonic vibrations will not affect the ability of the thermocouple 600 to measure the voltage difference across the two dissimilar metals (waveguide 154 and metal 500).

FIG. 6 shows another example of a waveguide 454 according to the present disclosure wherein a series of metals 500a, 500b, and 500c are deposited therealong. Metals 500a, 500b, and 500c are disposed at node points along the waveguide 454 to avoid asymmetry along the waveguide 454 (or the formation of an imbalance) during activation or propagation of the ultrasonic wave. If the layers are sufficiently thin, asymmetry may be avoided other issues may become relevant, contacts 510 rubbing through metal 500.

Turning back to FIG. 6, metal 500a is deposited across node A, metal 500b is deposited across node B, and metal 500c is deposited across node C. Each metal 500a, 500b, and 500c is, in turn, connected to a respective electrical potential of thermocouple 600 via a respective lead, 510a, 510b, and 510c. The proximal end 454a of the waveguide 454 connects to a different electrical potential of the thermocouple 600 via second lead 520. By virtue of the dissimilar metals 500a, 500b, and 500c deposited atop the waveguide 454 at the various nodes A, B, and C (which metals 500a, 500b, and 500c may be all the same or all different depending upon a particular purpose) and the unique characteristics of the Seebeck effect, the respective temperature at each node A, B and C compared across the temperature at the proximal end 454 measured by lead 520 may be reliably measured.

As can be appreciated, this will allow the surgeon to monitor the temperature along the waveguide 454 as the waveguide 454 both heats during activation and, more importantly, cools after activation. Knowing the temperature of the portion of the waveguide 454 at metals 500a, 500b, and 500c allows the surgeon to monitor safety, storage, or handling. Once again, ultrasonic vibrations will not affect the ability of the thermocouple 600 to measure the voltage difference across the dissimilar metals (waveguide 154 and metals 500a, 500b, 500c).

In aspects, the thermocouple 600 may be utilized with additional circuitry or algorithms that analyze the energy/heat flow along the waveguide 154, 454 as the energy/heat propagates proximally from the blade 162 over time by incorporating such variables as the specific heat and mass of the waveguide 154, 454 which could be useful with advancements in instrument control.

Moreover, disposing one or more metals 500a, 500b, and 500c to act as thermocouples on a node A, B, and C may have additional advantages. For example, placing a metal, metal 500a, to act as a thermocouple on a node, e.g., node A, may be used to monitor frequency change during activation. Additional metals 500b and 500c acting as thermocouples may be used to track frequency information across several nodes A, B and C or between nodes.

Moreover, knowing the temperature of the nodes along the waveguide 154, 454 may have additional other benefits as well. For example, by knowing the temperature of the various nodes A, B and C along the waveguide 154, 454 or at any point along the waveguide as well as knowing the temperature of the transducer 140 and the change in frequency (along or between the nodes A, B and C), it is possible to estimate the temperature of the blade 162. Additional thermocouples may need to be added for accuracy. This estimate could be used alone for measuring blade temperature or in conjunction with the above-mentioned embodiment wherein the metal is place directly on the blade 162 to measure the temperature using the Seebeck effect and act as a secondary verification for measuring blade temperature.

While several aspects of the disclosure have been detailed above and are shown in the drawings, it is not intended that the disclosure be limited thereto, as it is intended that the disclosure be as broad in scope as the art will allow and that the specification be read likewise. For example, the surgical instrument 100 may be part of a larger surgical system 10 (See FIG. 1) all configured to connect to a communication hub either directly or in a wireless manner. As such, the temperature information of the waveguide 154, 454 may be monitored by the surgeon on the instrument 100, for example, display panel 185 (FIG. 1) or at a point remote from the instrument 100, for example, panel 240 on the generator 200.

In aspects, the housing 112 may include circuitry 113 that communicates with the display panel 185 to convert the voltage obtained from the temperature difference between the first and second metals (waveguide 154, 454 and metal 500 (or metals 500a, 500b, 500c)) to an actual temperature on the waveguide 154, 454 proximate the metal 500 (or metals 500a, 500b, 500c), the circuitry communicating and the display panel 240 displaying the temperature in real time.

Other types of systems are envisioned such as those described in commonly-owned U.S. Patent Application Ser. No. 63/343,231 entitled SURGICAL SYSTEM INCLUDING A CORDLESS SURGICAL INSTRUMENT COMMUNICATION HUB, AND ONE OR MORE CONNECTED DEVICES filed May 18, 2022—the entire contents of which being incorporated by reference herein.

Therefore, the above description and accompanying drawings should not be construed as limiting, but merely as exemplifications of particular aspects. Those skilled in the art will envision other modifications within the scope and spirit of the claims appended hereto.

What is claimed is:

1. An ultrasonic surgical instrument, comprising:
a housing including an elongated shaft extending therefrom;
an ultrasonic transducer including a waveguide operably coupled thereto and configured to extend through the elongated shaft, the ultrasonic transducer adapted to couple to an energy source to allow selective activation thereof;
an ultrasonic blade made from a first metal operably coupled to the waveguide and configured to extend therefrom, the ultrasonic blade configured to vibrate upon activation of the ultrasonic transducer to treat tissue;
a second, dissimilar metal disposed on a portion of the ultrasonic blade; and
a thermocouple formed by a first electrical lead extending from a reference junction at the proximal end portion of the waveguide and a second electrical lead coupled to the second, dissimilar metal, the thermocouple configured to detect a temperature difference between the proximal end portion of the waveguide and the portion of the blade supporting the second, dissimilar metal, the thermocouple deriving a temperature at the second, dissimilar metal from a voltage generated at a junction between the first metal and the second, dissimilar metal and a temperature at the reference junction.

2. The ultrasonic surgical instrument according to claim 1, wherein the second, dissimilar metal is deposited onto the blade via metal or plasma deposition, chemical vapor deposition, printing, spraying, sintering, curing, thermal spray (HVOF), 3D printing, silk screen, or applying metal inks or paints.

3. The ultrasonic surgical instrument according to claim 1, wherein the ultrasonic instrument includes a handle operably coupled to the housing and configured to selectively move relative thereto to pivot a jaw member relative to the ultrasonic blade to clamp tissue therebetween.

4. The ultrasonic surgical instrument according to claim 1, wherein the housing includes circuitry that converts the voltage obtained from the temperature difference to an actual temperature on the blade at the junction with the second, dissimilar metal, the circuitry communicating with a display panel disposed on the housing for displaying the temperature.

5. The ultrasonic surgical instrument according to claim 4, wherein the circuitry converts the voltage and communicates to the display panel in real time.

6. An ultrasonic surgical instrument, comprising:
a housing including an elongated shaft extending therefrom;
an ultrasonic transducer including a waveguide made from a first metal operably coupled thereto and configured to extend through the elongated shaft, the ultrasonic transducer adapted to couple to an energy source to allow selective activation thereof;
an ultrasonic blade operably coupled to the waveguide and configured to extend therefrom, the ultrasonic blade configured to vibrate upon activation of the ultrasonic transducer to treat tissue;
a plurality of second, dissimilar metals disposed along the waveguide and extending proximally from the ultrasonic blade; and
a thermocouple formed by a first electrical lead extending from a reference junction operably associated with the housing and a second electrical lead coupled to each second, dissimilar metal, the thermocouple configured to detect a temperature difference between the reference junction and the portion of the blade supporting each second, dissimilar metal, the thermocouple deriving a temperature at each of the plurality of second, dissimilar metals from a voltage generated at a junction between the first metal and each of the plurality of second, dissimilar metals and a temperature at the reference junction.

7. The ultrasonic surgical instrument according to claim 6, wherein the plurality of second, dissimilar metals is deposited onto the waveguide via metal or plasma deposition, chemical vapor deposition, printing, spraying, sintering, curing, thermal spray (HVOF), 3D printing, silk screen, or applying metal inks or paints.

8. The ultrasonic surgical instrument according to claim 6, wherein the ultrasonic instrument includes a handle operably coupled to the housing and configured to selectively move relative thereto to pivot a jaw member relative to the ultrasonic blade to clamp tissue therebetween.

9. The ultrasonic surgical instrument according to claim 6, wherein the housing includes circuitry that converts the voltage obtained from the temperature difference to an actual temperature on the waveguide at each junction on the waveguide supporting the plurality of second, dissimilar metals, the circuitry communicating with a display panel disposed on the housing for displaying the temperature.

10. The ultrasonic surgical instrument according to claim 9, wherein the circuitry converts the voltage and communicates to the display panel in real time.

11. The ultrasonic surgical instrument according to claim 6, wherein at least one of the plurality of second, dissimilar metals is disposed on a node of the waveguide.

12. The ultrasonic surgical instrument according to claim 6, wherein each of the plurality of second, dissimilar metals is disposed on a node of the waveguide.

13. A method of determining a temperature of a blade of an ultrasonic surgical instrument, comprising:
electrically coupling a thermocouple across a first electrical lead extending from a proximal end portion of a waveguide and integrally associated with an ultrasonic blade made from a first metal and a second electrical lead coupled to a second, dissimilar metal disposed atop the ultrasonic blade;
obtaining a reference temperature at a reference junction;
activating a transducer to energize the waveguide to vibrate the ultrasonic blade to treat tissue proximate the ultrasonic blade;
utilizing the thermocouple to detect a temperature difference between the proximal end portion of the waveguide and the portion of the blade supporting the second, dissimilar metal on the blade, the thermocouple deriving a temperature at the second, dissimilar metal from a voltage generated at a junction between the first metal and the of second, dissimilar metal and the reference junction; and
utilizing the temperature of the portion of the blade supporting the second, dissimilar metal for at least one of safety, storage, or handling.

14. The method of determining a temperature of a blade of an ultrasonic surgical instrument according to claim 13, further comprising: depositing the second, dissimilar metal atop the blade via metal or plasma deposition, chemical vapor deposition, printing, spraying, sintering, curing, thermal spray (HVOF), 3D printing, silk screen, or applying metal inks or paints.

15. The method of determining a temperature of a blade of an ultrasonic surgical instrument according to claim 13, wherein a housing includes circuitry that includes the thermocouple, the circuitry configured to convert the voltage obtained from the temperature difference to an actual temperature on the blade at the portion of the blade supporting the second, dissimilar metal and wherein the method further comprises: communicating with a display panel disposed on the housing for displaying the temperature.

16. The method of determining a temperature of a blade of an ultrasonic surgical instrument according to claim 15, wherein the circuitry converts the voltage and communicates to the display panel in real time.
